(19) United States
(12) Patent Application Publication
(10) Pub. No.: US 2022/0273611 A1
(43) Pub. Date: Sep. 1, 2022

(54) ORONASAL CBD FORMULATIONS AND USES THEREOF

(71) Applicant: Healthaide Inc., San Diego, CA (US)

(72) Inventors: Tony Hugli, San Diego, CA (US); Glynn Wilson, Jupiter, FL (US)

(73) Assignees: Healthaide Inc., San Diego, CA (US); Jupiter Wellness, Inc., Jupiter, FL (US)

(21) Appl. No.: 17/739,104

(22) Filed: May 7, 2022

Related U.S. Application Data
(63) Continuation of application No. 17/353,573, filed on Jun. 21, 2021.
(60) Provisional application No. 63/042,458, filed on Jun. 22, 2020.

(51) Int. Cl. A61K 31/352; A61K 9/00; A61K 9/08; A61K 9/12; A61K 31/047; A61K 31/05; A61K 31/164; A61K 31/197; A61K 31/198; A61K 31/573; A61K 31/727

(52) U.S. Cl. CPC A61K 31/352; A61K 9/0014; A61K 9/0043; A61K 9/0075; A61K 9/008; A61K 9/08; A61K 9/124; A61K 31/047; A61K 31/05; A61K 31/164; A61K 31/197; A61K 31/198; A61K 31/573; A61K 31/727

(57) ABSTRACT

Provided herein are oronasal formulations including a cannabinoid and N-L-alpha-aspartyl-L-phenylalanine 1-methyl ester (APM). Also provided are methods of using the formulations.

### BACKGROUND

Viral infections commonly affect the upper or lower respiratory tract. Viruses that cause respiratory illnesses include coronaviruses (e.g., SARS-CoV-2), influenza viruses, rhinoviruses, and respiratory syncytial virus. SARS-CoV-2 is a respiratory virus that can cause an acute respiratory syndrome, COVID-19, which can be fatal. Respiratory viruses such as SARS-CoV-2 are often transmitted through droplets inhaled through the nose or mouth. Wearing a fabric mask can help reduce the risk of transmission of certain respiratory viruses. However, additional methods of reducing the transmission of respiratory viruses such as SARS-CoV-2 are needed.

### BRIEF SUMMARY

The present disclosure provides an oronasal formulation comprising a cannabinoid and N-L-alpha-aspartyl-L-phenylalanine 1-methyl ester (APM) or a lower alkyl derivative thereof.

In another aspect, the present disclosure provides a method of treating a viral infection in a mammal, comprising administering to the mammal an effective amount of a composition comprising a cannabinoid and N-L-alpha-aspartyl-L-phenylalanine 1-methyl ester (APM) or a lower alkyl derivative thereof. The method may include prophylactically treating the viral infection.

In another aspect, the present disclosure provides a method for supporting immune health in a mammal, comprising administering to the mammal an effective amount of a composition comprising a cannabinoid and N-L-alpha-aspartyl-L-phenylalanine 1-methyl ester (APM) or a lower alkyl derivative thereof.

Other objectives, advantages and novel features of the disclosure will become more apparent from the following detailed description.

### DETAILED DESCRIPTION

Presented herein are oronasal formulations comprising a cannabinoid and N-L-alpha-aspartyl-L-phenylalanine 1-methyl ester (APM) or a lower alkyl derivative thereof. Also presented are methods for treating viral infection, and for supporting immune health. In certain embodiments, the oronasal formulations are used in such methods.

In the present description, the term “about” means±20% of the indicated range, value, or structure, unless otherwise indicated. The term “consisting essentially of ” limits the scope of a claim to the specified materials or steps and those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include” and “have” are used synonymously, which terms and variants thereof are intended to be construed as non-limiting. The term “comprise” means the presence of the stated features, integers, steps, or components as referred to in the claims, but that it does not preclude the presence or addition of one or more other features, integers, steps, components, or groups thereof. Any ranges provided herein include all the values and narrower ranges in the ranges.

### Cannabinoids

Provided herein are formulations and uses of formulations that include a cannabinoid and an N-L-alpha-aspartyl-L-phenylalanine 1-methyl ester (APM) or a lower alkyl derivative thereof.

The term “cannabinoid” refers to a class of compounds that bind to one or more cannabinoid receptors and act on the endocannabinoid system. Cannabinoids include phytocannabinoids, endocannabinoids, and non-naturally occurring cannabinoids. The endocannabinoid system is a biological system present in mammals that includes endocannabinoids, which are lipid based neurotransmitters that bind to cannabinoid receptors. Cannabinoid receptor 1(CB1) and cannabinoid receptor 2 (CB2) are expressed in the central and peripheral nervous system, and cannabinoid receptor 3 (CB3) is expressed is the central nervous system. Other non-classical cannabinoid receptors include G protein-coupled receptor (GPR55), GRP119 and GPR18, peroxisome proliferator-activated receptors (PPARs) and transient receptor potential vanilloid 1 (TRPV1).

Endocannabinoid signaling through cannabinoid receptors affect cognitive processes such as mood, appetite, and memory. Cannabinoids are also present on a variety of other cells types and tissues. For example, CB2 is expressed on monocytes, macrophages, and B and T cells.

In certain embodiments, the cannabinoid is a phytocannabinoid. A phytocannabinoid is a cannabinoid that is naturally produced by a plant. Phytocannabinoids are typically C21 or C22 (for the carboxylated forms) terpenophenolic compounds. Plants that produce cannabinoids include Cannabis, Echinacea purpurea, Echinacea angustifolia, Acmelia oleracea, Helichrysum umbraculigerum, and Radula marginata. Examples of phytocannabinoids include dodeca-2E, 4E, 8Z, 10E/Z-tetraneoic-acid-isobutyl amid, beta-caryophyllene, perottetinene, Δ9-Tetrahydrocannabinol (THC), cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA), cannabielsoin (CBE), and cannabicitran (CBT).

In certain embodiments, the phytocannabinoid comprises a Cannabis-derived phytocannabinoid. Cannabis generally refers to the plant genus that includes Cannabis sativa, Cannabis sativa forma indica, and Cannabis ruderalis. Examples of phytocannabinoids produced by Cannabis include Δ9-Tetrahydrocannabinol (THC), cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), tetrahydrocannabivarinic acid (THCVA), cannabielsoin (CBE), and cannabicitran (CBT) (see, e.g., Prandi et al., Molecules 23(7), 1526, 2018). Cannabis-derived cannabinoids accumulate in secretory cavities of trichomes, which are present in the female flower of the plant. Cannabinoids may also be present in lower concentrations in seeds, roots, and stems of the plant. Many cannabis strains have either THCA or CBDA as the predominant cannabinoid produced, although it is typical for a variety of cannabinoids to be present together. When THCA and CBDA are decarboxylated, such as through heat treatment, the molecules are converted to THC and CBD, respectively.

In certain embodiments, the cannabis-derived phytocannabinoid comprises CBD. In some embodiments, the cannabis-derived phytocannabinoid comprises CBD and at least one other cannabis-derived phytocannabinoid.

In certain embodiments, the cannabinoid comprises an endocannabinoid. Endocannabinoids are lipid-based neurotransmitters that are endogenously expressed and bind to cannabinoid receptors of the endocannabinoid system. Examples of endocannabinoids include anandamide, arachidonoyl-ethanolamide (AEA), 2-arachidonoyl-glycerol (2-AG), 2-arachidonyl glyceryl ether (noladin ether), N-arachidonoyl domain (NADA), virodhamine (OAE), and lysophosphatidylinositol (LPI). In certain embodiments, the endocannabinoid comprises anandamide.

In certain specific embodiments, the cannabinoid comprises a non-naturally occurring cannabinoid (also referred to as “synthetic cannabinoid”). Examples of non-naturally occurring cannabinoids include CP55,940, which is a potent THC mimic; WIN 55,212-2 (which is an aminoalkylindole derivative with cannabinoid receptor agonist activity), nabilone (which is structurally very similar to THC), JWH-018 (1-pentyl-3-(1-naphthoyl)indole), dimethylheptylpyran, HU-210 (which is about 100 times as potent as THC), HU-331 (which is a quinone anticancinogenic drug synthesized from cannobidiol), JWH-133 (which is a potent selective CB2 receptor agonist), Levonantradol (Nantrodolum), or AM-2201 (which is a potent cannabinoid receptor agonist). In certain particular embodiments, the synthetic cannabinoid comprises CP55,940, WIN 55,212-2, or nabilone.

### N-L-alpha-aspartyl-L-phenylalanine 1-methyl ester

As previously noted, provided herein are formulations and uses of formulations that include a cannabinoid and an N-L-alpha-aspartyl-L-phenylalanine 1-methyl ester (APM) or a lower alkyl derivative thereof. N-L-alpha-aspartyl-L-phenylalanine 1-methyl ester (APM) may also be referred to as aspartame.

The term “lower alkyl derivative of APM” refers to a compound where the methyl group of the 1-methyl ester of APM is replaced with an alkyl group having 2-4 carbons, such as ethyl, propyl, isopropyl, or butyl.

### Formulations

Provided herein are formulations comprising a cannabinoid and N-L-alpha-aspartyl-L-phenylalanine 1-methyl ester (APM) or a lower alkyl derivative thereof. Such formulations include pharmaceutical formulations (also referred to as “pharmaceutical compositions”) and non-pharmaceutical formulations (also referred to “non-pharmaceutical compositions”). Proper formulation is dependent upon the route of administration chosen. Any acceptable techniques, carriers, and excipients are suitable to formulate the formulations described herein; such as those described in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999). A pharmaceutical formulation refers to a formulation for use in the treatment for a disease, disorder or condition, or for treating one or more symptoms of the disease, disorder or condition. A non-pharmaceutical formulation refers to a formulation other than a pharmaceutical formulation, such as a dietary supplement and a nutraceutical formulation.

In certain embodiments, the cannabinoid is provided in the formulation in the form of a cannabinoid isolate. The term “cannabinoid isolate” refers to a highly purified cannabis-derived cannabinoid. A cannabinoid isolate may be produced, for example, by CO₂ extraction, ethanol extraction, or butane extraction. Physical forms of a cannabinoid isolate include, for example, a crystal, a powder, a wax, or a resin. A cannabinoid isolate may have a total cannabinoid content of at least 65%, at least 70%, at least 75%, at least 80%, at least 85%, at least 90%, or at least 95% cannabinoid (w/v). In certain embodiments, the cannabinoid isolate has a total cannabinoid content of at least 95% (w/v). In certain embodiments the cannabinoid isolate has a total cannabidiol (CBD) content of at least 98% (w/v).

In some embodiments, the cannabinoid is provided in the formulation at about 0.01% to about 0.5% weight by volume (w/v). In certain embodiments, the cannabinoid is provided in the formulation at about 0.025% to about 0.5% (w/v). In certain embodiments, the cannabinoid is provided in the formulations at about 0.01% to about 0.05%, about 0.05% to about 0.1%, about 0.1% to about 0.2%, about 0.2% to about 0.3%, about 0.3% to about 0.4%, or about 0.4% to about 0.5% (w/v). Preferably, the cannabinoid is at a concentration of about 0.02% to about 0.5% (w/v), or about 0.25% to about 0.4% (w/v).

In some embodiments, the concentration of the N-L-alpha-aspartyl-L-phenylalanine 1-methyl ester (APM) or a lower alkyl derivative is about 0.01% to about 2% (w/v). In some embodiments, the concentration of the N-L-alpha-aspartyl-L-phenylalanine 1-methyl ester (APM) or a lower alkyl derivative is about 0.02 to about 1% (w/v). In some embodiments, the concentration of the N-L-alpha-aspartyl-L-phenylalanine 1-methyl ester (APM) or a lower alkyl derivative is about 0.01% to about 0.05%, about 0.05% to about 0.1%, about 0.1% to about 0.2%, about 0.2% to about 0.3%, about 0.3% to about 0.4%, or about 0.4% to about 0.5% (w/v). Preferably, the APM or lower alkyl derivative is at a concentration of about 0.02% to about 1%, or about 0.02 to about 0.5% (w/v).

In some embodiments, the ratio of the N-L-alpha-aspartyl-L-phenylalanine 1-methyl ester or the lower alkyl derivative thereof to the cannabinoid in the formulation is in the range of about 4:1 to about 10:1 (by weight). In some embodiments, the ratio of the N-L-alpha-aspartyl-L-phenylalanine 1-methyl ester or the lower alkyl derivative thereof to the cannabinoid in the formulation is about 2:1 to about 4:1, about 4:1 to about 5:1, about 5:1 to about 6:1, about 6:1 to about 7;1, about 7:1 to about 8:1, about 8:1 to about 9:1, or about 9:1 to about 10:1 (by weight). In some embodiments, the ratio of the N-L-alpha-aspartyl-L-phenylalanine 1-methyl ester or the lower alkyl derivative thereof to the cannabinoid in the formulation is in the range of about 5:1 to about 8:1 (by weight). As used herein, “carrier” and “physiologically acceptable carriers” are used interchangeably and include any and all solvents, buffers, dispersion media, coatings, surfactants, antioxidants, preservatives (e.g., antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art and are molecular entities and compositions that are generally non-toxic to recipients at the dosages and concentrations employed, i.e., do not produce an adverse, allergic or other untoward reaction when administered to an animal, such as a human, as appropriate (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in pharmaceutical or non-pharmaceutical formulations provided herein is contemplated.

In certain embodiments, the carrier is suitable to be included in oronasal formulations. Any suitable concentration of a single carrier or a combination of carriers can be employed. Typically, the suitable amount of carrier will depend upon the specific carrier(s) employed. Preferred concentration range of a single carrier or the total of a combination of carriers can be from about 0.1% to about 70%, more preferably from about 5.0% to about 30%, more specifically from about 10% to about 25% of the formulation. In certain particular embodiments, the carrier may include sodium chloride, microcrystalline cellulose, carboxymethylcellulose sodium, dextrose, dehydrated alcohol, lecithin, oelic acid, lactose monohydrate, anhydrous lactose, or any combination thereof.

The formulations may comprise different types of carriers depending on whether it is to be administered in solid, liquid or aerosol form. The formulations as describe herein (and any additional active agent) can be administered intranasally, mucosally, orally, topically, locally, by inhalation (e.g., aerosol inhalation), or by other methods or any combination of the forgoing as would be known to one of ordinary skill in the art (see, for example, Remington's Pharmaceutical Sciences, 18th Ed. Mack Printing Company, 1990, incorporated herein by reference).

In certain embodiments, the formulation is an oronasal formulation. As used herein “oronasal” can refer to a route of administration into the nose, such as through one or both nasal passages, and/or the mouth, but is not intended to include drugs that are intended to be absorbed through the digestive tract. Oronasal delivery may include delivery to the airways, such as to the bronchial passages or lungs, for example, by inhalation. Oronasal delivery may also include delivery to the mucous membranes of the nasal passages, mouth, and/or throat. An oronasal formulation can be in the form of, for example, solutions, suspensions, gels, pastes, medicated sticks, balms, spray, powders (e.g., for a dry powder inhaler), creams or ointments. Such formulations optionally contain carriers, emollients, absorption enhancing agents, solubilizers, stabilizers, tonicity enhancing agents, buffers, preservatives, propellants, and/or additional therapeutic agents.

In embodiments, the formulation (preferably the oronasal formulation) may include an absorption enhancing agent. An absorption enhancing agent refers to an agent that that functions to increase absorption by enhancing membrane permeation. In certain particular embodiments, the absorption enhancing agent dimethyl isosorbide, diethylene glycol monoethyl ether, or both. Examples of concentration ranges that the absorption enhancing agents may be provided in include about 0.1% to about 10%, or about 0.5% to about 5% (w/v).

In embodiments, the formulation (preferably the oronasal formulation) may include a preservative that exhibits antimicrobial properties. For example, preservatives can be present in a gelled formulation to minimize bacterial and/or fungal over its shelf-life. Non-limiting examples for use herein include diazolidinyl urea, methylparaben, propylparaben, butylparaben, isobutylparaben, tetrasodium EDTA, and ethylparaben. The preservative may include a combination of parabens, such as methylparaben and propylparaben. In certain specific embodiments, the preservative is merfen and thiomersal; stabilized chlorine dioxide; and quaternary ammonium compounds such as benzalkonium chloride, cetyltrimethylammonium bromide and cetylpyridinium chloride, sorbic acid, paraben, phenoxyethanol, caprylyl glycol, ethylhexylglycerin, hexylene glycol or a combination thereof. In certain embodiments, the preservative is selected from sorbic acid, benzalkonium chloride, phenylcarbinol, phenylethyl alcohol, or a combination thereof. Examples of concentration ranges that the preservatives may be provided in include about 0.1% to about 10%, or about 0.5% to about 5% (w/v).

A formulation (e.g., an oronasal formulation) of a cannabinoid and N-L-alpha-aspartyl-L-phenylalanine 1-methyl ester (APM) or a lower alkyl derivative can contain one or more “lipophilic solvent(s).” A lipophilic solvent can be miscible with water and/or lower chain alcohols and have a vapor pressure less than water at 25° C. (˜23.8 mm Hg). A lipophilic solvent can be a glycol, specifically propylene glycol. In particular, the propylene glycol can be from the class of polyethylene glycols, specifically polyethylene glycols ranging in molecular weight from 200 to 20000. A lipophilic solvent can be from the class of glycol ethers, such as diethylene glycol monoethyl ether (transcutol, or 2-(2-ethoxyethoxy)ethanol {CAS NO 001893} or ethyoxydiglycol).

In some embodiments, the formulations (e.g., oronasal formulations) are in the form of a suspension containing one or more polymers as suspending agents. Example polymers include water-soluble polymers such as cellulosic polymers, e.g., hydroxypropyl methylcellulose, and water-insoluble polymers such as cross-linked carboxyl-containing polymers. Certain formulations described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the formulations (e.g., oronsal formulations) are in the form of a dry powder and include a powdered carrier, such as lactose powder.Examples of lactose powders suitable as dry powder carriers include lactose monohydrate and anhydrous lactose.

In some embodiments, the formulations (e.g., oronasal formulations) include solubilizing agents to aid in the solubility of the cannabinoids and/or the N-L-alpha-aspartyl-L-phenylalanine 1-methyl ester (APM) or a lower alkyl derivative. The term “solubilizing agent” generally includes agents that result in formation of a micellar solution or a true solution of the agent. Certain acceptable nonionic surfactants, for example polysorbate 80, are useful as solubilizing agents. Examples include glycols, polyglycols, e.g., polyethylene glycol 400, and glycol ethers.

In certain embodiments, the formulations (e.g., oronasal formulations) include an additional active agent. The additional active agent, may for example have antiviral effects. Examples of other additional active agents that may be included in the formulations include Aloe vera leaf extra, xylitol, an anticoagulant such as ethylenediaminetetraacetic acid (EDTA) or heparin, and dexamethasone.

A formulation (e.g., an oronasal formulation) of a cannabinoid and N-L-alpha-aspartyl-L-phenylalanine 1-methyl ester (APM) or a lower alkyl derivative can also contain a gelling agent that increases the viscosity of the final solution. The gelling agent can also act as an emulsifying agent. The formulations can form clear gels and soft gels, which upon application such as in the nose or mouth can break down and deteriorate. Typically, the concentration and combination of gelling agents will depend on the physical stability of the finished product. Preferred concentration range of a gelling agent can be from about 0.01% to about 20%, more preferably from about 0.1% to about 10%, more specifically from about 0.5% to about 5% of the formulation (w/v). Non-limiting examples for use herein include classes of celluloses, acrylate polymers and acrylate crosspolymers. Preferably, hydroxypropyl cellulose, hydroxymethyl cellulose, Pluronic PF127 polymer, carbomer 980, carbomer 1342 and carbomer 940, more preferably hydroxypropyl cellulose, Pluronic PF127 carbomer 980 and carbomer 1342, more specifically hydroxypropyl cellulose (Klucel® EF, GF and/or HF), Pluronic PF127, carbomer 980 and/or carbomer 1342 (Pemulen® TR-1, TR-2 and/or Carbopol® ETD 2020).

A formulation (e.g., an oronasal formulation) of a cannabinoid and N-L-alpha-aspartyl-L-phenylalanine 1-methyl ester (APM) or a lower alkyl derivative can contain one or more anti-oxidants, thiol containing compounds radical scavengers, and/or stabilizing agents, preferred concentration range from about 0.001% to about 0.1%, more preferably from about 0.1% to about 5% of the formulation (w/v). Non-limiting examples for use herein include butylatedhydroxytoluene, butylatedhydroxyanisole, ascorbyl palmitate, citric acid, vitamin E, vitamin E acetate, vitamin E-TPGS, ascorbic acid, sodium metabisulfite, tocophersolan and propyl gallate. More specifically the anti-oxidant can be ascorbyl palmitate, vitamin E acetate, vitamin E-TPGS, vitamin E or butylatedhydroxytoluene.

A formulation (e.g., an oronasal formulation) of a cannabinoid and N-L-alpha-aspartyl-L-phenylalanine 1-methyl ester (APM) or a lower alkyl derivative can optionally include one or more chelating agents. As used herein, the term “chelating agent” or “chelator” refers to those agents capable of removing a metal ion from a system by forming a complex so that the metal ion cannot readily participate in or catalyze chemical reactions. The chelating agents for use herein are preferably formulated at concentrations ranging from about 0.001% to about 10%, more preferably from about 0.05% to about 5.0% of the formulation (w/v). Non-limiting examples for use herein include EDTA, disodium edeate, dipotassium edeate, cyclodextrin, trisodium edetate, tetrasodium edetate, citric acid, sodium citrate, gluconic acid and potassium gluconate. Preferably, the chelating agent can be EDTA, disodium EDTA, dipotassium EDTA, trisodium EDTA or potassium gluconate.

In some embodiments, the oronasal formulations are topically applied to the mucous membranes within the nose or mouth. Such topically administered formulations can be provided in any suitable form, preferably as a gel, a lotion, or a cream, but also in an ointment or oil base, as well as a sprayable liquid form (e.g., an oral or nasal spray that includes the cannabinoid and N-L-alpha-aspartyl-L-phenylalanine 1-methyl ester (APM) or a lower alkyl derivative in a base, vehicle or carrier). In some particular embodiments, the topically administered oronasal formulation is formulated as a gel. In other particular embodiments, the topically administered oronasal formulation is formulated as an ointment.

In certain embodiments, a formulation (e.g., an oronasally administered formulation) including a cannabinoid and N-L-alpha-aspartyl-L-phenylalanine 1-methyl ester (APM) or a lower alkyl derivative has a pH of about 4 to about 7.5. In certain embodiments, the formulation has a pH of about 4 to about 4.5, about 4.5 to about 5, about 5 to about 5.5, about 5.5 to about 6, about 6 to about 6.5, or about 6.5 to about 7. Preferably, the formulation has a pH in the range of about 5.0 to about 7.0, such as about 5.5 to about 6.5.

In some embodiments, the formulations (e.g., oronasal formulations) include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the formulation in an acceptable range.

In some embodiments, the formulations (e.g., oronasal formulations) include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some embodiments, the formulations (e.g., oronasal formulations) include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

In certain embodiments, a formulation including a cannabinoid and N-L-alpha-aspartyl-L-phenylalanine 1-methyl ester (APM) or a lower alkyl derivative may be administered orally. A formulation of the invention to be orally administered can be prepared by combining a cannabinoid and N-L-alpha-aspartyl-L-phenylalanine 1-methyl ester (APM) or a lower alkyl derivative with an appropriate pharmaceutically acceptable carrier, diluent or excipient by standard methods known to one skilled in the art. The oral formulation may be in the form of liquid, tablets, powders, gels, syrups, elixirs, slurries, suspensions and the like. For example, an oral formulation may be in the form of a liquid spray used to coat the mucous membranes of the mouth and throat.

In some embodiments, the formulations including a cannabinoid and N-L-alpha-aspartyl-L-phenylalanine 1-methyl ester (APM) or a lower alkyl derivative are formulated for administration by inhalation. The formulations may be inhaled through the nose or the mouth. Various forms suitable for administration by inhalation include, but are not limited to, aerosols, mists and powders. Formulations of the cannabinoid and N-L-alpha-aspartyl-L-phenylalanine 1-methyl ester (APM) or a lower alkyl derivative may be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator is formulated containing a powder mix of the cannabinoid and N-L-alpha-aspartyl-L-phenylalanine 1-methyl ester (APM) or a lower alkyl derivative, and a suitable powder base such as lactose or starch.

In certain embodiments, the inhalable formulation is in the form of a nasal spray. A nasal spray may include the oronasal formulation packaged into a bottle or similar container that is capable of emitting the formulation in a liquid stream or mist. The formulation may be emitted using the pressure of ambient air, for example, by use of a pump sprayer, of a flexible container that can be squeezed to emit the formulation. Alternatively, the formulation may be emitted from a pressurized container that is pressurized with an inert gas, such as nitrogen, argon, or carbon dioxide.

Formulations including the cannabinoid and N-L-alpha-aspartyl-L-phenylalanine 1-methyl ester (APM) or a lower alkyl derivative described herein may be manufactured by means of conventional mixing, dissolving, emulsifying, entrapping or lyophilizing processes. Formulations may be formulated in conventional manner using one or more physiologically acceptable carriers, diluents, excipients or auxiliaries which facilitate processing of the formulations into preparations that can be used pharmaceutically. Proper formulation is dependent upon the route of administration chosen.

Oronasal formulations disclosed herein may be prepared by (a) mixing hydrophilic ingredients and water to form a 1^{st} mixture, (b) mixing hydrophobic ingredients to form a 2^{nd} mixture, and (c) mixing the 1^{st} mixture and the 2^{nd} mixture together to form a 3^{rd} mixture. In step (a), AMP or a lower alkyl derivative thereof may be mixed with other hydrophilic ingredients together. Preferably, AMP is added after the other hydrophilic ingredients are already mixed together. In step (b), cannabinoid may be mixed with other hydrophobic ingredients together. Preferably, cannabinoid is added after the other hydrophobic ingredients are already mixed together. In certain other embodiments, cannabinoid may be added to the 3^{rd} mixture in a further step, step (d). Optionally, additional ingredients (e.g., a thickening agent) may be added the mixture that comprises both AMP or a lower alkyl derivative and cannabinoid in a further step, step (e).

In certain embodiments, an oronasal formulation is prepared by combining and mixing hydrophilic ingredients (e.g., glycerin, dimentyl isosorbide, glycereth-7, PEG-100 stearate, phenoxyethanol, methylparaben, ethylparaben, propylparaben, butylparaben, isobutylparaben, Aloe vera leaf extract (100×), hydroxypropyl starch phosphate, and/or polysorbate 20) with water. The aqueous mixture may be stirred and heated, such as at 65-80 degrees Celsius, preferably at 70-72 degrees Celsius. APM at an appropriate concentration (e.g., within the range of about 0.2% to about 2% (w/v)) may be then mixed with the aqueous mixture and stirred until dissolved. The resulting mixture (“the 1st mixture”) may similarly be heated again (if necessary).

Hydrophobic ingredients (e.g., isocetyl stearate, arlacel 165, isocetyl palmitate, Jojoba oil, tridecyl stearate, tridecyl trimellitate, dipentaerythrityl hexacaprylate/hexacaprate, PEG-7, cetearyl alcohol, ceteareth-20, cetyl ricinoleate, and/or stearic acid) may be combined and mixed. The mixture (“the 2nd mixture”) may be also stirred and heated, such as at 65-80 degrees Celsius (I., 70-72 degrees Celsius), and kept at the appropriate temperature until the mixture becomes clear and homogenous.

Next, CBD at an appropriate concentration (e.g., 0.01-0.5% (w/v)) may be mixed with the 2^{nd} mixture and then added to the 1^{st} mixture to form a composition comprising both cannabinoid and APM. Alternatively, CBD may be added after the 2^{nd} mixture is mixed with the 1^{st} mixture. The mixing of the 1^{st} mixture with the 2^{nd} mixture is preferably performed with rapid agitation, such as using a propeller stirrer, but without formation of a vortex.

The composition comprising both cannabinoid and APM should be stirred until the temperature cools to 60 degrees Celsius. At this temperature, the mixing should be switched to high shear mixing, such as with a homomixer. Next, a thickening agent (e.g., polyacrylamide (and) C13-14 isoparaffin (and) laureth-7; 1.5%) may be slowly added. High shear mixing may be continuous for an appropriate period of time, and the mixing may be switched to a gate-type mixer. The mixing may be continued until the mixture cools to a temperature of between 25 and 30 degrees Celsius. The appearance of the resulting formulation is preferably white, glossy, and viscous; the pH is preferably in the range of 4.9 and 5.5; the specific gravity is preferably in the range of 0.97 and 0.99; and the water content is preferably in the range of 50% to 61%.

Sterilization or adequate antimicrobial preservation may be included in methods of producing the oronasal formulations. Since formulations of the present invention are intended to be administered intranasally or intraorally, it is preferred that they be free of pathogenic organisms. A benefit of a sterile liquid suspension is that it reduces the possibility of introducing contaminants into the individual when the suspension formulation is administered, thereby reducing the chance of an opportunistic infection. Processes which may be considered for achieving sterility may include any appropriate sterilization steps known in the art. In one embodiment, the active agents are produced or isolated under sterile conditions, the processing is performed in a sterile environment, and the packaging is conducted under sterile conditions. Alternatively, the formulations may be sterile filtered and filled in containers providing sterile formulations which are used in a nasal spray device or inhaler, for example. In certain embodiments, one or more ingredients in the present formulation may be sterilized by steam, gamma radiation or prepared using or mixing sterile steroidal powder and other sterile ingredients where appropriate. Additionally, the formulations may be prepared and handled under sterile conditions, or may be sterilized before or after packaging.

### Methods of Use

Provided herein are methods of using formulations comprising a cannabinoid and N-L-alpha-aspartyl-L-phenylalanine 1-methyl ester (APM) or a lower alkyl derivative thereof as previously described.

“Mammal” includes humans and both domestic animals such as laboratory animals and household pets, (e.g., cats, dogs, swine, cattle, sheep, goats, horses, rabbits), and non-domestic animals such as wildlife and the like. In certain specific embodiments, the mammal is a human. In certain specific embodiments, the mammal is a pet, such as a dog or cat.

A “subject” according to any of the above embodiments is a mammal. Mammals include but are not limited to, domesticated animals (e.g., cows, sheep, cats, dogs, and horses), primates (e.g., human and non-human primates such as monkeys), rabbits, and rodents (e.g., mice and rats). Preferably the subject is a human. In certain embodiments, the subject does not have phenylketonuria. In some embodiments, the subject is at risk of contracting a viral infection, such as COVID-19.

“Treatment,” “treating” or “ameliorating” refers to medical management of a condition, disease, or disorder of a subject (e.g., patient) to reduce or eliminate a symptom, reduce the duration, or delay onset or progression of the condition, disease, or disorder.

An “effective amount” refers to an amount of a formulation including a cannabinoid and N-L-alpha-aspartyl-L-phenylalanine 1-methyl ester (APM) or a lower alkyl derivative thereof that provides a desired physiological change, such as an antiviral effect. In certain embodiments, the effective amount is a prophylactically effective amount. In certain embodiments, the effective amount is a therapeutically effective amount. The desired physiological change may, for example, be a decrease in symptoms of a disease, or a decrease in severity of the symptoms of the disease, or may be a reduction in the progression of symptoms of the disease. The desired physiological change may include relief from irritation, discomfort, pain, or inflammation, such as rhinitis or sore throat. In certain embodiments, the desired physiological change does not involve treatment of a disease.

In certain embodiments, the methods include treating a viral infection in a mammal, comprising administering to the mammal an effective amount of a composition comprising a cannabinoid and N-L-alpha-aspartyl-L-phenylalanine 1-methyl ester (APM) or a lower alkyl derivative thereof. Treating a viral infection may include shortening the duration of the infection, reducing the severity of the infection, and/or alleviating one or more symptoms of the viral infection.

In certain embodiments, the methods include supporting immune health of a mammal, comprising administering to the mammal an effective amount of a composition comprising a cannabinoid and N-L-alpha-aspartyl-L-phenylalanine 1-methyl ester (APM) or a lower alkyl derivative thereof “Supporting immune health” as used herein refers to promoting resistance to pathogens, such as reducing the rate and/or severity of entry into host cells by pathogenic bacteria, viruses, and fungi. In some embodiments, formulations described herein are used for supporting immune health of cells of the oronasal passages. Supporting immune health of a mammal may include supporting antiviral immunity. “Supporting antiviral immunity” as used herein refers to promoting resistance of a cell, a tissue, or a mammal to viruses. In some embodiments, supporting antiviral immunity includes blocking a virus from entry into host cells. In some embodiments, formulations described herein are useful for supporting antiviral immunity of cells of the oronasal passages. Supporting antiviral immunity may be evidenced by a decrease in rate of infection or severity of infection despite viral exposure.

In some embodiments, the viral infection is caused by a respiratory virus. Respiratory viruses are viruses that enter the oral and/or nasal passages and infect the respiratory system. Common symptoms of infection by respiratory viruses include fever, sore throat, coughing, and nasal congestion. Examples of respiratory viruses include coronaviruses, influenza viruses, rhinoviruses, and respiratory syncytial virus. In particular embodiments, the respiratory virus comprises a coronavirus, an influenza virus, a rhinovirus, and respiratory syncytial virus.

In particular embodiments, the respiratory virus is a betacoronavirus. Betacoronaviruses that have been known to infect humans include EMC/2012, SARS-CoV-1, and SARS-CoV-2. Severe cases of infection with betacoronaviruses may result in acute respiratory distress syndrome, which is characterized by rapid onset of inflammation in the lungs. Betacoronaviruses have been shown to rely on the receptor TMPRSS2 for infection. In particular embodiments, the methods of treating viral infection include inhibiting TMPRSS2-dependent entry of betacoronaviruses. In particular embodiments, the respiratory virus is SARS-CoV-2. SARS-CoV-2 is the virus that causes the infection known as COVID-19. Symptoms of COVID-19 may include fever, chill, cough, shortness of breath, difficulty breathing, fatigue, muscle or body aches, headache, loss of taste or smell, sore throat, nasal congestion, nausea, vomiting, and/or diarrhea.

In particular embodiments, the treating comprises prophylactically treating. In such embodiments, the mammal may not exhibit symptoms of viral infection at the time of administering. The prophylactically treating may result in a decreased risk of infection for the mammal who has received the cannabinoid and the APM, as compared to a control mammal who has not received the cannabinoid and the APM. The prophylactically treating may result in a shortened duration of infection of a decreased severity of infection, if the mammal becomes infected, as compared to a control mammal who has not received the cannabinoid and the APM. A subject to be prophylactically treated includes a mammal (e.g., a human) that may be or have been exposed to a viral pathogen.

In embodiments, the administering comprises oronasally administering.

In some embodiments, the administering comprises oral inhalation. Oral inhalation may be a useful route of administration to deliver the formulation deeper into the airway, for example, to the bronchials and/or lungs. Administering by oral inhalation may be performed, for example, using a metered-dose inhaler, a dry powder inhaler, or a liquid spray bottle.

In some embodiments, the administering comprises nasal inhalation. Nasal inhalation may be a useful form of administration, for example, to deliver the formulation deep into the nasal sinuses. Administering by nasal inhalation may be performed, for example, using a nasal spray, a nasal dry powder device, or a pipette.

In some embodiments, the administering comprises topically administering to the mouth or throat. Administering topically to the mouth or throat may be performed, for example, using a liquid such as a mouthwash, a liquid spray bottle, an ointment, or a gel.

In some embodiments, the administering comprises topically administering to one or both nasal passages. Administering topically to nasal passages may be performed, for example, using a liquid spray bottle, an ointment, or a gel. In some embodiments, the formulation is a solution for use as a nasal lavage.

The appropriate dosage of the cannabinoid and N-L-alpha-aspartyl-L-phenylalanine 1-methyl ester (APM) or a lower alkyl derivative thereof (used alone or in combination with one or more other additional therapeutic agents) will depend on the type of disease or condition, the route of administration, body weight of the subject, severity and progression of the disease, whether the formulation is administered for preventive or therapeutic purposes, previous or concurrent therapeutic interventions, the subject's clinical history and response to the cannabinoid and N-L-alpha-aspartyl-L-phenylalanine 1-methyl ester (APM) or a lower alkyl derivative thereof, and the discretion of the attending physician. The practitioner responsible for administration will be able to determine the concentration of active ingredient(s) in a composition and appropriate dosing for the subject to be treated. Various dosing schedules including but not limited to single or multiple administrations over various time-points, bolus administration, and pulse infusion are contemplated herein.

The cannabinoid and N-L-alpha-aspartyl-L-phenylalanine 1-methyl ester (APM) or a lower alkyl derivative thereof may be used in an amount effective to achieve the intended purpose. For use to treat or prevent a disease condition, the cannabinoid and N-L-alpha-aspartyl-L-phenylalanine 1-methyl ester (APM) or a lower alkyl derivative thereof, or formulations thereof, are administered in a therapeutically effective amount. Determination of a therapeutically effective amount is within the capabilities of those of skill in the art, especially in light of the details provided herein.

In certain embodiments, the daily dosage of the formulation including the cannabinoid and N-L-alpha-aspartyl-L-phenylalanine 1-methyl ester (APM) or a lower alkyl derivative thereof ranges from about 0.1 μg/kg to about 100 mg/kg or more of the cannabinoid, and about 0.1 μg/kg to about 100 mg/kg or more of the N-L-alpha-aspartyl-L-phenylalanine 1-methyl ester (APM) or a lower alkyl derivative thereof. For repeated administrations over several days or longer, depending on the condition, the treatment may be sustained until a desired suppression of disease symptoms occurs or a risk of contracting the disease occurs. In some embodiments, a single dose of a formulation includes a range from about 0.001 mg/kg to about 10 mg/kg of the cannabinoid and about 0.0001 to about 100mg/kg of the N-L-alpha-aspartyl-L-phenylalanine 1-methyl ester (APM) or a lower alkyl derivative thereof.

In some embodiments, a single dose (e.g., a metered inhaler dose or a single spray from a nasal spray bottle) may include 1-50 mg of CBD per administration.

In some embodiments, a dose may include 0.5-50 mg of N-L-alpha-aspartyl-L-phenylalanine 1-methyl ester (APM) or a lower alkyl derivative thereof per administration.

In some embodiments, an oral dose may include about 5 μg/kg/body weight to about 25 μg/kg/body weight, about 25 μg/kg/body weight to about 50 μg/kg/body weight, about 50 μg/kg/body weight to about 250 μg/kg/body weight, or about 250 μg/kg/body weight to about 500 μg/kg/body weight of cannabinoid per administration, and any range derivable therein.

In some embodiments, an oral dose may include about 5 μg/kg/body weight to about 25 μg/kg/body weight, about 25 μg/kg/body weight to about 50 μg/kg/body weight, about 50 μg/kg/body weight to about 250 μg/kg/body weight, or about 250 μg/kg/body weight to about 500 μg/kg/body weight of N-L-alpha-aspartyl-L-phenylalanine 1-methyl ester (APM) or a lower alkyl derivative thereof per administration.

Such doses may be administered intermittently, e.g., 2-3 times per day, every week, or every three weeks. An initial higher loading dose, followed by one or more lower doses may be administered. However, other dosage regimens may also be used.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 63/042,458, filed on Jun. 22, 2020, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. An oronasal formulation, comprising a cannabinoid and N-L-alpha-aspartyl-L-phenylalanine 1-methyl ester (APM) or a lower alkyl derivative thereof, wherein:
(i) the cannabinoid is at a concentration of about 0.01% to about 0.5% weight by volume (w/v), preferably about 0.02% to about 0.5% (w/v); and
(ii) the N-L-alpha-aspartyl-L-phenylalanine 1-methyl ester or lower alkyl derivative thereof to the cannabinoid in the formulation is in the range of about 4:1 to about 10:1 (by weight).

2. The oronasal formulation according to claim 1, wherein the cannabinoid comprises a phytocannabinoid, an endocannabinoid, or a non-naturally occurring cannabinoid.

3. The oronasal formulation according to claim 1, wherein the cannabinoid is a phytocannabinoid.

4. The oronasal formulation according to claim 3, wherein the phytocannabinoid comprises a cannabis-derived phytocannabinoid.

5. The oronasal formulation according to claim 4, wherein the cannabis-derived phytocannabinoid comprises one or more of .DELTA.9-Tetrahydrocannabinol (THC), cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), tetrahydrocannabivarinic acid (THCVA), cannabiolsoin (CBE), and cannabicitran (CBT).

6. The oronasal formulation according to claim 4, wherein the cannabis-derived phytocannabinoid comprises CBD.

7. The oronasal formulation according to claim 4, wherein the cannabis-derived phytocannabinoid comprises a cannabinoid isolate having a total cannabinoid content of at least 95% cannabinoid (w/v).

8. The oronasal formulation according to claim 1, wherein the cannabinoid comprises an endocannabinoid.

9. The oronasal formulation according to claim 8, wherein the endocannabinoid comprises anandamide.

10. The oronasal formulation according to claim 1, wherein the cannabinoid comprises a non-naturally occurring cannabinoid.

11. The oronasal formulation according to claim 10, wherein the non-naturally occurring cannabinoid comprises CP55,940, WIN 55,212-2, or nabilone.

12. (canceled)

13. (canceled)

14. (canceled)

15. The oronasal formulation according to claim 1, further comprising a carrier.

16. The oronasal formulation according to claim 15, wherein the carrier comprises sodium chloride, microcrystalline cellulose, carboxymethylcellulose sodium, dextrose, dehydrated alcohol, lecithin, oelic acid, lactose monohydrate, anhydrous lactose, or any combination thereof.

17. The oronasal formulation according to claim 1, further comprising a preservative.

18. The oronasal formulation according to claim 17, wherein the preservative comprises sorbic acid, phenylcarbinol, phenylethyl alcohol, ethanol, or any combination thereof.

19. The oronasal formulation according to claim 1, further comprising an additional active agent.

20. The oronasal formulation according to claim 19, wherein the additional active agent comprises xylitol, dexamethasone, heparin, or ethylenediaminetetraacetic acid (EDTA).

21. The oronasal formulation according to claim 1, further comprising a propellant.

22. The oronasal formulation according to claim 21, wherein the propellant comprises hydrofluoroalkane.

23. The oronasal formulation according to claim 1, wherein the formulation has a pH of about 4.5 tp about 7.5.

24. The oronasal formulation according to claim 1, wherein the formulation is in the form of a liquid, a gel, or a powder.

25. A spray bottle comprising an oronasal formulation according to claim 1.

26. (canceled)

27. (canceled)

28. An inhaler comprising an oronasal formulation according to claim 1.

29. (canceled)

30. (canceled)

31. A mouthwash comprising an oronasal formulation according to claim 1.

32. A liquid solution for nasal lavage comprising an oronasal formulation according to claim 1.

33. A method of treating a viral infection in a mammal, comprising administering to the mammal an effective amount of a composition comprising a cannabinoid and N-L-alpha-aspartyl-L-phenylalanine 1-methyl ester (APM) or a lower alkyl derivative thereof, wherein:
(i) the cannabinoid in the composition is at a concentration of about 0.01% to about 0.5% weight by volume (w/v), preferably about 0.02% to about 0.5% (w/v); and
(ii) the N-L-alpha-aspartyl-L-phenylalanine 1-methyl ester or lower alkyl derivative thereof to the cannabinoid in the formulation is in the range of about 4:1 to about 10:1 (by weight).

34. The method according to claim 33, wherein the viral infection is caused by a respiratory virus.

35. The method according to claim 34, wherein the respiratory virus comprises a coronavirus, an influenza virus, a rhinovirus, or respiratory syncytial virus.

36. The method according to claim 34, wherein the respiratory virus comprises a betacoronavirus.

37. The method according to claim 36, wherein the respiratory virus comprises SARS-CoV-2.

38. The method according to claim 33, wherein the treating comprising prophylactically treating.

39. A method of supporting immune health in a mammal comprising administering to the mammal an effective amount of a composition comprising a cannabinoid and N-L-alpha-aspartyl-L-phenylalanine 1-methyl ester (APM) or a lower alkyl derivative thereof, wherein:
(i) the cannabinoid in the composition is at a concentration of about 0.01% to about 0.5% weight by volume (w/v), preferably about 0.02% to about 0.5% (w/v); and
(ii) the N-L-alpha-aspartyl-L-phenylalanine 1-methyl ester or lower alkyl derivative thereof to the cannabinoid in the formulation is in the range of about 4:1 to about 10:1 (by weight).

40. The method according to claim 33, wherein the administering comprises oronasally administering.

41. The method according to claim 33, wherein the administering comprises oral inhalation.

42. The method according to claim 33, wherein the administering comprises nasal inhalation.

43. The method according to claim 33, wherein the administering comprises topically administering to the mouth and/or throat.

44. The method according to claim 33, wherein the administering comprises topically administering to one or both nasal passages.

45. The method according to claim 33, wherein the mammal is a human.

46. (canceled)

47. The method according to claim 33, wherein the cannabinoid comprises an endocannabinoid, a phytocannabinoid, or a non-naturally occurring cannabinoid.

48. The method according to claim 47, wherein the cannabinoid is a phytocannabinoid.

49. The method according to claim 48, wherein the phytocannabinoid cannabinoid is a cannabis-derived phytocannabinoid.

50. The method according to claim 49, wherein the cannabis-derived phytocannabinoid comprises one or more of .DELTA.9-Tetrahydrocannabinol (THC), cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), tetrahydrocannabivarinic acid (THCVA), cannabielsoin (CBE), and cannabicitran (CBT).

51. The method according to claim 50, wherein the cannabis-derived phytocannabinoid comprises CBD.

52. The method according to claim 49, wherein the cannabis-derived phytocannabinoid comprises a cannabinoid isolate having a total cannabinoid content of at least 95% cannabinoid (w/v).

53. The method according to claim 47, wherein the cannabinoid comprises an endocannabinoid.

54. The method according to claim 53, wherein the endocannabinoid comprises anandamide.

55. The method according to claim 47, wherein the cannabinoid comprises a non-naturally occurring cannabinoid.

56. The method according to claim 47, wherein the non-naturally occurring cannabinoid comprises CP55,940, WIN 55,212-2, or nabilone.
